(19) United States
(12) Patent Application Publication
(10) Pub. No.: US 2024/0307039 A1
(43) Pub. Date: Sep. 19, 2024

(54) ULTRASOUND PHANTOM AND METHOD OF PRODUCING ULTRASOUND PHANTOM

(71) Applicant: CANON KABUSHIKI KAISHA, Tokyo (JP)

(72) Inventors: KYOHEI WADA, Tokyo (JP); CHIAKI NISHIURA, Tokyo (JP); RYO OGAWA, Kanagawa (JP)

(21) Appl. No.: 18/598,091

(22) Filed: Mar. 7, 2024

(30) Foreign Application Priority Data
Mar. 13, 2023 (JP) 2023-039061
Feb. 27, 2024 (JP) 2024-027845

(51) Int. Cl. A61B 8/00

(52) U.S. Cl. CPC A61B 8/587

(57) ABSTRACT

Provided is an ultrasound phantom including: water; a polysaccharide (A) having a sol-gel phase transition point of 30° C. or more and 95° C. or less; and a polysaccharide thickener (B) whose 1 mass % aqueous solution has a viscosity at room temperature of 300 mPa·s or more, wherein the ultrasound phantom is configured to show a tan δ of 0.2 or more under a gel state, which is obtained by dynamic viscoelasticity measurement at 1 Hz and room temperature.

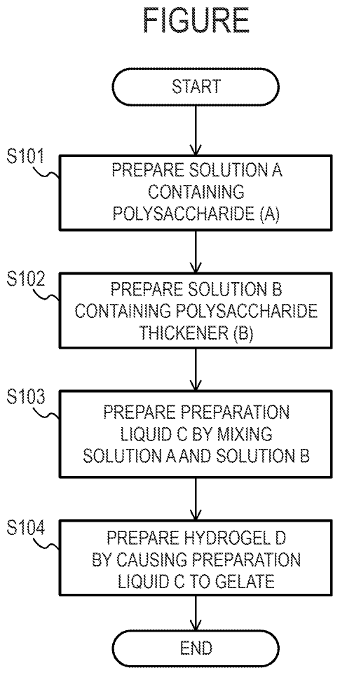

### BACKGROUND OF THE INVENTION

### Field of the Invention

The present invention relates to an ultrasound phantom and a method of producing an ultrasound phantom.

### Description of the Related Art

A phantom simulating a living organism tissue or the like has been used in the calibration of an apparatus for medical diagnosis and the implementation of its function. Examples of the apparatus for medical diagnosis include an acoustic wave (e.g., an ultrasonic wave) diagnostic apparatus, a magnetic resonance imaging diagnostic apparatus (MRI), a computed tomographic apparatus (CT), an X-ray diagnostic apparatus, and a near-infrared imaging apparatus (NIRI). Each of those apparatus utilizes an ultrasonic wave or an electromagnetic wave, such as an X-ray or light, and monitors a physical phenomenon, such as the scattering, refraction, reflection, absorption, diffraction, or interference of such wave, to diagnose a disease of a patient.

In recent years, in the use of an ultrasonic diagnostic apparatus, the quantitative evaluation of the hardness (Young's modulus) of an organ by a shear wave elastography method (SWE method) has become widespread. The SWE method is a method including observing the propagation velocity of a shear wave generated by an ultrasonic wave to calculate a Young's modulus in the organ, and enables the quantitative evaluation of its hardness. Further, in recent years, there has been a growing demand for the measurement of a viscous property in addition to the hardness (Young's modulus) of the organ. An ultrasound phantom having a predetermined Young's modulus and a predetermined viscous property is used in calibration for calculating the accurate viscoelasticity of the organ with the ultrasonic diagnostic apparatus.

With regard to a viscosity characteristic, in this specification, the viscosity characteristic of a polysaccharide thickener to be used as a component for a composition is referred to as “viscosity”, and the viscosity characteristic of a phantom in a gel state is referred to as “viscous property” so that the viscosity characteristics may be distinguished from each other.

As an approach to controlling the viscous property of an ultrasound phantom, in Japanese Patent No. 6,754,112, there is a disclosure of a method including using glycerin.

However, when glycerin is used as a component for the ultrasound phantom, glycerin has an increasing effect on a sound speed as compared to that in a glycerin-free ultrasound phantom. Accordingly, when glycerin is used in a large amount as a component for the ultrasound phantom for improving its viscous property, a divergence may occur between the sound speed in the ultrasound phantom and a sound speed in a living organism. In addition, when the phantom is produced while the content of glycerin is adjusted so as to match the acoustic characteristic of the living organism, the width of the viscous property to be obtained is small, and hence it is difficult to control the viscous property to desired viscoelasticity.

### SUMMARY OF THE INVENTION

The present invention has been made in view of the above-mentioned problems. That is, an object of the present invention is to provide an ultrasound phantom that can have a high viscous property while suppressing a divergence from a sound speed in a living organism.

According to one aspect of the present invention, there is provided an ultrasound phantom including: water; a polysaccharide (A) having a sol-gel phase transition point of 30° C. or more and 95° C. or less; and a polysaccharide thickener (B) whose 1 mass % aqueous solution has a viscosity at room temperature of 300 mPa·s or more, wherein the ultrasound phantom is configured to show a tan δ of 0.2 or more under a gel state, which is obtained by dynamic viscoelasticity measurement at 1 Hz and room temperature.

According to another aspect of the present invention, there is provided a method of producing an ultrasound phantom, the method including: preparing a solution A containing a polysaccharide (A) having a sol-gel phase transition point of 30° C. or more and 95° C. or less; preparing a solution B containing a polysaccharide thickener (B) whose 1 mass % aqueous solution has a viscosity at room temperature of 300 mPa·s or more; preparing a preparation liquid C by mixing the solution A and the solution B; and preparing a hydrogel D by causing the preparation liquid C to gelate, wherein the hydrogel D in a gel state shows a tan δ of 0.2 or more, which is obtained by performing dynamic viscoelasticity measurement at 1 Hz and room temperature.

Further features of the present invention will become apparent from the following description of exemplary embodiments with reference to the attached drawing.

### BRIEF DESCRIPTION OF THE DRAWINGS

FIGURE is a flowchart for illustrating the outline of a method of producing an ultrasound phantom.

### DESCRIPTION OF THE EMBODIMENTS

An ultrasound phantom according to the present invention includes: water; a polysaccharide (A) having a sol-gel phase transition point of 30° C. or more and 95° C. or less; and a polysaccharide thickener (B) whose 1 mass % aqueous solution has a viscosity at room temperature of 300 mPa·s or more. In addition, the ultrasound phantom according to the present invention shows a tan δ of 0.2 or more under a gel state, which is obtained by dynamic viscoelasticity measurement at 1 Hz and room temperature.

The ultrasound phantom is hereinafter also simply referred to as “phantom”.

The term “room temperature” as used herein refers to a temperature of 24.9° C. or more and 25.1° C. or less.

### Polysaccharide (A) Having Sol-Gel Phase Transition Point of 30° C. or More and 95° C. or Less

The polysaccharide (A) is a polysaccharide having a sol-gel phase transition point of 30° C. or more and 95° C. or less, and is a polysaccharide that is in a gel state at room temperature. Whether the polysaccharide is in a sol state or a gel state is measured with a dynamic viscoelasticity-measuring apparatus such as MCR302 manufactured by Anton Paar Japan K.K.

In the present invention, the term “gel” refers to a solid state, and the term “sol” refers to a state having fluidity. Whether the polysaccharide is in a gel state or a sol state can be judged by performing, for example, dynamic viscoelasticity measurement. Specifically, the storage modulus and loss modulus of the polysaccharide are determined by dynamic viscoelasticity measurement at 0.5 Hz and room temperature, and the value of the tan δ thereof, which corresponds to a ratio obtained by dividing the storage modulus by the loss modulus, is calculated. When the value of the tan δ is less than 1, the polysaccharide can be judged to be gel, and when the value of the tan δ is 1 or more, the polysaccharide can be judged to be sol.

Gel formed of the polysaccharide (A) is important for the control of the storage modulus and tan δ of the phantom because the gel has a rigid molecular structure. That is, when the phantom includes the polysaccharide (A), the phantom hardens to exhibit a suppressing effect on the fluidity of the polysaccharide thickener (B) to be described later. Accordingly, as the content of the polysaccharide (A) in the phantom becomes larger, the tan δ of the phantom tends to be smaller.

In the present invention, to secure the uniformity of the phantom, a polysaccharide that is sensitized to temperature to be brought into a gel state may be used as the polysaccharide (A). Examples of the polysaccharide that is sensitized to temperature to be brought into a gel state include agar, curdlan, and a gellan gum.

Agar is a mucinous substance extracted from a red alga, such as an agar weed or Chinese moss, and is a polysaccharide containing at least agarose and agaropectin. The production area, origin, and the like of the agar to be used as the polysaccharide (A) are not limited, and any agar may be used. To adjust the strength of gel, so-called low-strength agar (e.g., low-strength agar as disclosed in Japanese Patent Application Laid-Open No. H05-317008) that has undergone molecular cleavage through acid treatment may be used as the agar.

The agar has the first phase transition point at which the agar solates through an increase in temperature thereof, and the second phase transition point at which the agar gelates through a reduction in temperature thereof. In general, the first phase transition point is 85° C. or more and 99° C. or less, and the second phase transition point is 30° C. or more and 45° C. or less.

The agar is generally a hydrogel material that becomes sol by being heated to 85° C. or more in water, and undergoes a structural transition by being cooled to 45° C. or less, to thereby become gel. When the agar is in a sol state, the entanglement of polymer chains in the agar is released, and hence the agar liquefies. Meanwhile, when the agar is cooled, the polymer chains in the agar form a double-helix structure, and as the cooling proceeds, the chains form higher order entanglement to cause the agar to gelate. Via the foregoing gelation mechanism, the Young's modulus of the phantom correlates with the concentration of the agar, and hence the phantom can be adjusted to various kinds ranging from soft gel to hard gel. The phantom has been required to simulate various organs, and hence required Young's moduli cover a wide range. The agar may be suitably used in the adjustment of the Young's modulus.

When the total mass of the water and a sound speed-adjusting agent to be described later is set to 100 parts by mass, the blending amount of the agar in the phantom is preferably 0.01 part by mass or more and 20 parts by mass or less, and to obtain a uniform phantom free of any air bubble or the like, the blending amount is more preferably 0.01 part by mass or more and 10 parts by mass or less, still more preferably 0.01 part by mass or more and 0.5 part by mass or less. The adjustment of the blending amount of the agar in the ranges enables the production of phantoms having various viscoelasticities. When the blending amount of the agar in the phantom is 0.01 part by mass or more, the value of the tan δ of the phantom can be controlled. In addition, when the blending amount of the agar in the phantom is 20 parts by mass or less, a significant increase in viscosity of a composition for producing the phantom by the agar that has been thermally dissolved can be avoided, and hence it becomes easier to obtain a uniform phantom.

The curdlan is described in, for example, the Journal of the Japanese Society for Food Science and Technology, Vol. 38, No. 8, 736-742 (1991), and is one kind of polysaccharide produced by a microorganism (Alcaligenes faecalis var. myxogenes, or many strains of Agrobacterium, or Rhizobium). The only sugar for forming the curdlan is D-glucose, and 99% or more of its glucoside bonds are β-1,3 bonds. The curdlan is insoluble in water, but is soluble in an alkaline aqueous solution of sodium hydroxide or the like.

A method including adding water to curdlan powder and vigorously stirring the mixture with, for example, a high-speed homogenizer or a cutter mixer has been known as a method of preparing a uniform aqueous dispersion liquid of the curdlan. In addition, a method including adding the curdlan to warm water at about 55° C. while stirring the warm water with, for example, a hand or a propeller stirrer, and then cooling the mixture has been known as another method of preparing a uniform aqueous dispersion liquid of the curdlan. The heating of the aqueous dispersion liquid of the curdlan prepared by such method forms gel.

The gel obtained by the heating is roughly classified into two types depending on its treatment temperature. That is, the two types are thermally irreversible gel obtained by heating at 80° C. or more, and thermally reversible gel obtained by heating at about 60° C. and then cooling, and are referred to as “high-set gel” and “low-set gel,” respectively. In addition, gel may be prepared as follows without heating: the curdlan is dissolved in an alkaline aqueous solution, and while the solution is left at rest, the solution is neutralized with a carbon dioxide gas or the like, or sodium hydroxide is removed with a dialysis membrane. Alternatively, gel may be prepared by adding a cation, such as a calcium or magnesium ion, to the alkaline aqueous solution to form a crosslinked structure of a dissociated hydroxy group and the cation.

When the total mass of the water and a sound speed-adjusting agent to be described later is set to 100 parts by mass, the blending amount of the curdlan in the phantom is preferably 0.1 part by mass or more and 10 parts by mass or less. In addition, to obtain a uniform phantom free of any air bubble or the like, the blending amount is more preferably 0.1 part by mass or more and 5 parts by mass or less. When the blending amount of the curdlan in the phantom is 0.1 part by mass or more, the value of the tan δ of the phantom can be controlled. In addition, when the blending amount of the curdlan in the phantom is 10 parts by mass or less, a uniform phantom can be obtained.

The gellan gum is produced by separating and purifying a polysaccharide accumulated by a microorganism Sphingomonas elodea, which is collected from a water plant, in the outside of the cells of the bacterium through use of glucose or the like as a nutrient. The gellan gum comes in the following two kinds: a HA gellan gum containing a high concentration of an acyl group, and a LA gellan gum from which an acyl group is removed. In the present invention, each of the gellan gums may be appropriately used.

The HA gellan gum containing a high concentration of an acyl group has a dissolving temperature of 85° C., and hence enables the production of opaque gel that is soft and firm like gelatin.

The LA gellan gum containing a low concentration of an acyl group has a dissolving temperature of 90° C., and hence enables the production of transparent gel that is hard and brittle like agar.

When the total mass of the water and a sound speed-adjusting agent to be described later is set to 100 parts by mass, the blending amount of the gellan gum in the phantom is preferably 0.1 part by mass or more and 10 parts by mass or less. In addition, to obtain a uniform phantom free of any air bubble or the like, the blending amount is more preferably 0.1 part by mass or more and 5 parts by mass or less. When the blending amount of the gellan gum in the phantom is 0.1 part by mass or more, the value of the tan δ of the phantom can be controlled. In addition, when the blending amount of the gellan gum in the phantom is 10 parts by mass or less, a uniform phantom can be obtained.

The agar or the gellan gum, which has a low setting temperature and is excellent in handleability, is preferably used as the polysaccharide (A), and the agar is particularly preferably used.

### Polysaccharide Thickener (B)

The polysaccharide thickener (B) to be used in the present invention is in a sol state in the gel of the above-mentioned polysaccharide (A) at 35° C. or less, and has a tan δ of 1 or more in dynamic viscoelasticity measurement at 0.5 Hz and room temperature.

The polysaccharide thickener (B) exhibits strong stickiness in the gel of the phantom to contribute to the viscous property of the phantom.

The polysaccharide thickener (B) that may be used in the present invention is the following polysaccharide: a solution obtained by adding the polysaccharide to water so that its concentration may be 1 mass % has a viscosity at room temperature of 300 mPa·s or more. The use of such polysaccharide as a thickener provides a phantom having a specifically high tan δ value. Thus, the Young's modulus and viscous property of the phantom in a gel state in the present invention can be controlled.

The viscosity of the solution, which is obtained by adding the polysaccharide thickener (B) to water so that its concentration may be 1 mass %, at room temperature may be measured with a rotational viscometer. Specifically, the viscosity may be measured by, for example, using a dynamic viscoelasticity-measuring apparatus MCR302 (manufactured by Anton Paar Japan K.K.) and parallel plates each having a diameter of 25 mm at a rotational speed of 5 (/s).

A polysaccharide thickener having a mannose residue in the main chain thereof, and having a galactose residue in a side chain thereof may be used as the polysaccharide thickener (B). The ratio of the number of the galactose residues in the side chain to the number of the mannose residues in the main chain is preferably 0.2 or more and 0.4 or less.

The polysaccharide thickener having the mannose residue in the main chain, and having the galactose residue in the side chain may further have a derived group. Examples of the derived group of the polysaccharide thickener having the mannose residue in the main chain, and having the galactose residue in the side chain include a hydroxypropyl group, a hydroxypropyltrimonium chloride group, a carboxymethyl group, a phosphoric acid group, a hydroxyethyl group, and a hydroxypropylmethyl group.

When the polysaccharide thickener having the mannose residue in the main chain, and having the galactose residue in the side chain further has the derived group, the entanglement of the main chain of the polysaccharide thickener (B) is easily released. Thus, the solubility of the polysaccharide thickener (B) is improved, and hence a uniform sol liquid of the polysaccharide thickener (B) can be obtained.

Specific examples of the polysaccharide thickener (B) include a guar gum, a locust bean gum, a tamarind seed gum, and a xanthan gum.

To increase the viscosity of a 1 mass % aqueous solution of the polysaccharide thickener (B) at room temperature, the selection of a molecular weight based on a grade is important, and in the case of, for example, a guar gum, a molecular weight of 90,000 or more is preferred, and a molecular weight of 900,000 or more is more preferred. The use of the polysaccharide thickener (B) having a high molecular weight increases the frequency at which the molecules of the polysaccharide are entangled with each other, to thereby contribute to an improvement in viscous property of the phantom.

The molecular weight of the polysaccharide thickener (B) can be measured by using a gel-permeation chromatography (GPC). As the GPC, for example, 1,200 series manufactured by Agilent Technologies can be used. For the measurement of the molecular weight, a pretreated sample which is prepared by diluting a sample with eluent to 0.1% by weight followed by filtering using a 0.45 μm multilayer filter can be used. Specific measurement conditions are, for example, as follows: column is TSKgel GMPWXL×2; eluent is 200 mM Sodium nitrate aqueous solution; flow rate is 1.0 mL/min; detector is an RI detector; and column temperature is 40° C. As standards of the molecular weight, for example, pullulan and glucose can be used. The measurement of the molecular weight by GPC is a statistical measurement for distributed objects, and it should be noted that the measured value obtained by GPC may be referred to as number average molecular weight.

When such polysaccharide thickener (B) as described above is used, the polysaccharide thickener (B) is present as sol in the gel of the polysaccharide (A) to exhibit stickiness, and hence the viscous property of the phantom in the present invention can be controlled.

When the total mass of the water and a sound speed-adjusting agent to be described later is set to 100 parts by mass, the blending amount of the polysaccharide thickener (B) in the phantom is preferably 0.5 part by mass or more and 20 parts by mass or less. When the blending amount of the polysaccharide thickener (B) in the phantom is 0.5 part by mass or more, a phantom showing a tan δ of 0.2 or more can be produced. When the blending amount of the polysaccharide thickener (B) in the phantom is 20 parts by mass or less, excessive entanglement of the molecules of the polysaccharide can be avoided. Thus, the fluidity of the polysaccharide thickener (B) in the phantom is maintained, and hence the viscous property of the phantom can be secured.

### Sound Speed-Adjusting Agent

In the present invention, a sound speed-adjusting agent may be used for adjusting a sound speed in the phantom. A compound that improves the water retentivity of the phantom is preferably selected as the sound speed-adjusting agent.

A compound having a polar term dP in Hansen solubility parameters (HSP) of 13 MPa^{0.5} or more is preferred as the compound that improves the water retentivity. The Hansen solubility parameters may be calculated with computer software “Hansen Solubility Parameters in Practice (HSPiP),” and may be determined by using a known method. The Hansen solubility parameters as described herein are values calculated by utilizing the version 5.3.05 of the HSPiP.

Such compounds each having a water retentivity-improving effect as described above may be collectively referred to as “chaotropic agents.” The chaotropic agents each have a changing effect on an interaction between water molecules in an aqueous solution. The addition of a molecule having a large polar term dP as a water retention aid to the aqueous solution changes the interaction between the water molecules, and hence the water retention aid can strongly attract the water molecules. The extent of the attraction is represented by the polar term dP, and hence the use of a molecule whose polar term has a large value as a sound speed-adjusting agent can achieve the stability of the phantom serving as a hydrogel with time.

For example, dimethyl sulfoxide (DMSO), urea, guanidine, or a guanidine salt may be used as the sound speed-adjusting agent satisfying the above-mentioned conditions. In addition, a derivative thereof, for example, a methylated, dimethylated, ethylated, or diethylated product thereof may also be used.

When the total mass of the water and the sound speed-adjusting agent is set to 100 parts by mass, the blending amount of the sound speed-adjusting agent in the phantom is preferably 1 part by mass or more. When the blending amount of the sound speed-adjusting agent is 1 part by mass or more, a sufficient sound speed-adjusting effect is obtained, and hence a divergence between the sound speed in the phantom and a sound speed in a living organism can be suppressed.

### Other Component

Various components may be used as required as other components in the phantom according to the present invention. For example, an ultrasonic scattering agent may be incorporated as any other component into the phantom. An ultrasonic diagnostic apparatus performs image taking and measurement with a signal that has reached its detector out of ultrasonic waves scattered in the phantom. Accordingly, when a portion serving as a measurement object in the phantom contains the ultrasonic scattering agent, an image of the portion can be taken, and hence the Young's modulus thereof can be measured.

In addition, the scattering efficiency of an ultrasonic wave is calculated by the acoustic impedance (=density×sound speed) of a substance scattering the wave. At a substance interface, the scattering efficiency becomes higher as a difference in acoustic impedance value between substances for forming the interface becomes larger.

A solid particle that may be used as the ultrasonic scattering agent is, for example, a metal particle, a metal oxide particle, a carbon particle, or a spherical polymer. A material for the ultrasonic scattering agent that may be used in the present invention is not particularly limited as long as the material is a solid having low water solubility. From the viewpoint of mechanical stability, the ultrasonic scattering agent is preferably, for example, a carbon crystal particle, such as graphite or micro diamond, a resin-made particle, such as a polyethylene particle, a polyethylene hollow sphere, or a polystyrene hollow sphere, an oxide fine particle, such as titanium oxide, alumina oxide, or silicon oxide, or a metal fine particle, such as tungsten, nickel, or molybdenum. Of those, a carbon crystal particle is particularly preferred in consideration of the magnitude of its acoustic impedance and its dispersibility in water.

The particle diameter of the ultrasonic scattering agent may be determined in accordance with the wavelength of an ultrasonic wave to be input. The particle diameter of the ultrasonic scattering agent calculated from the wavelength of an ultrasonic wave emitted from the probe of an ultrasonic diagnostic apparatus is preferably 5 μm or more and 50 μm or less.

However, in general, a high-density particle having a particle diameter of 5 μm or more has a high sedimentation rate, and hence separation occurs during the change of the agar from sol to gel. Its degree may be calculated from the following Stokes equation:

$$V = g ( ρ s - ρ 0 ) {d}^{2} / 18 η$$

where V represents the sedimentation rate, “g” represents a gravitational acceleration, ρs represents the density of the particles, ρ0 represents the density of a solvent, “d” represents the diameter of each of the particles, and η represents the viscosity of the solvent.

In the present invention, when the phantom includes the above-mentioned polysaccharide thickener (B), the value of η in the Stokes equation becomes larger, and hence the sedimentation of the ultrasonic scattering agent can be suppressed.

In addition, an antifungal agent may be added as any other component.

In general, fungi are liable to occur in a hydrogel, and hence when the hydrogel is used for calibration, the presence of the fungi may affect its physical property values.

Although the antifungal agent that may be used is not particularly limited, an antifungal agent having water solubility and a wide antimicrobial spectrum is preferred.

An antiseptic, a microbicidal agent, or an antimicrobial agent may be used as the antifungal agent. Specific examples of the antifungal agent include alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, oxyquinoline sulfate, phenethyl alcohol, and benzyl alcohol.

Of those, a parahydroxybenzoic acid ester-based compound is preferred from the viewpoint that the compound has water solubility and a wide antimicrobial spectrum, and in particular, an influence on a human body is small. In addition, methyl parahydroxybenzoate is particularly preferred from the viewpoint of water solubility.

The antifungal agent is preferably used while its blending amount in the phantom is appropriately adjusted because the respective compounds exhibit different effects. For example, when methyl parahydroxybenzoate is used as the antifungal agent, its blending amount is preferably 0.25 part by mass when the total mass of the water and the sound speed-adjusting agent is set to 100 parts by mass. When the blending amount of methyl parahydroxybenzoate is 0.25 part by mass, its effect as the antifungal agent can be highly obtained. When the blending amount of methyl parahydroxybenzoate is more than 0.25 part by mass, it becomes difficult to dissolve methyl parahydroxybenzoate in the phantom.

In addition, the phantom according to the present invention may include borax as any other component. Borax can chemically crosslink the polysaccharide thickener (B), and when the phantom includes borax, changes in viscosity and elastic modulus of the polysaccharide thickener (B) with time can be suppressed.

When the total mass of the water and the sound speed-adjusting agent is set to 100 parts by mass, the blending amount of borax in the phantom is preferably 0.001 part by mass or more and 0.2 part by mass or less. In addition, the blending amount of borax is more preferably 0.05 part by mass or more and 0.1 part by mass or less. When the blending amount of borax is 0.2 part by mass or less, an excessive reduction in value of the tan δ of the phantom due to an excessively large number of chemical crosslinking points can be alleviated, and hence the phantom can show a desired viscous property. In addition, when the blending amount of borax is 0.001 part by mass or more, borax can form a chemical crosslinkage to exhibit a high alleviating effect on the change of the polysaccharide thickener (B) with time.

In addition, the phantom according to the present invention may include an antifoaming agent as any other component. The entry of air bubbles at the time of the production of the hydrogel excessively scatters an ultrasonic wave owing to a difference in acoustic impedance at a water-air interface. Accordingly, the antifoaming agent is preferably used for suppressing the mixing of the air bubbles into the hydrogel.

Examples of the antifoaming agent that may be used in the phantom according to the present invention include: oils, such as a mineral oil, and oils and fats; surfactants, such as a fatty acid, a fatty acid ester, a phosphoric acid ester, and metal soap; and silicone compounds, such as a silicone oil and dimethylsiloxane. Of those, a silicone compound is particularly preferred as the antifoaming agent because a small blending amount thereof exhibits a high antifoaming effect.

It is preferred that the blending amount of the antifoaming agent in the phantom be appropriately adjusted because the respective compounds to be used as the antifoaming agents exhibit different effects. In the case of, for example, using the silicone compound as the antifoaming agent, when the mass of the water is set to 94.000 parts by mass, the compound only needs to be blended in an amount of 0.001 part by mass or more and 0.100 part by mass or less, and the blending can provide a sufficient effect as an antifoaming agent.

The ultrasound phantom of the present invention may further include, for example, a colorant such as an aqueous dye or a pH adjuster such as a phosphate buffer as any other component.

### Method of Producing Phantom

A flowchart for illustrating the outline of a method of producing an ultrasound phantom according to the present invention is illustrated in FIGURE.

The method of producing an ultrasound phantom according to the present invention includes the steps of: preparing a solution A containing the above-mentioned polysaccharide (A); preparing a solution B containing the above-mentioned polysaccharide thickener (B); preparing a preparation liquid C by mixing the solution A and the solution B; and preparing a hydrogel D by causing the preparation liquid C to gelate.

First, in Step S101, the solution A containing the above-mentioned polysaccharide (A) is prepared. A method of preparing the solution only needs to be appropriately set in accordance with characteristics corresponding to the kind of the polysaccharide (A) described above. For example, when agar is used as the polysaccharide (A), the solution A may be prepared by adding the agar to water, and then dissolving the agar therein through heating.

Next, in Step S102, the solution B containing the above-mentioned polysaccharide thickener (B) is prepared. A method of preparing the solution B may also be set in accordance with the characteristics of the polysaccharide thickener (B) to be used in the same manner as in the method of preparing the solution A. For example, when a guar gum is used as the polysaccharide thickener (B), the solution B may be prepared by adding the guar gum to water and stirring the mixture, followed by heating.

Subsequently, in Step S103, the solution A and the solution B are mixed so that the blending amounts of the polysaccharide (A) and the polysaccharide thickener (B) may each be a desired value, followed by sufficient stirring. Thus, the preparation liquid C is obtained. Uniform dispersion of the respective components in the preparation liquid C is important for securing the uniformity of the phantom. Whether or not the mixing and stirring of the solution A and the solution B in Step S103 have been enough to secure the uniformity of the phantom may be verified, for example, as described below. That is, when the inside of the resultant phantom is visualized with an ultrasonic diagnostic apparatus, the absence of a remarkable lump, haze, or the like only needs to be confirmed.

After that, in Step S104, the preparation liquid C obtained in Step S103 is loaded into a mold having a desired shape, and is caused to gelate to prepare the hydrogel D. Thus, the ultrasound phantom can be produced. Although a method of causing the preparation liquid C to gelate is not particularly limited, for example, when the solution A and the solution B are each prepared by heating, the following may be performed: the preparation liquid C is prepared under a state in which the solutions are each maintained at high temperature in Step S103, and the resultant preparation liquid C is cooled to be caused to gelate in Step S104.

The hydrogel D prepared in Step S104, which is in a gel state, shows a tan δ of 0.2 or more, which is obtained by performing dynamic viscoelasticity measurement at 1 Hz and room temperature. Such hydrogel D may be obtained by appropriately adjusting the blending amount of the polysaccharide (A) in the solution A and the blending amount of the polysaccharide thickener (B) in the solution B in accordance with the kinds of the respective materials to be used.

Step S101, that is, the step of preparing the solution A and Step S102, that is, the step of preparing the solution B may be performed in no particular order, and any one of the steps may be performed first.

The method of producing an ultrasound phantom according to the present invention may further include a step of preparing a solution E containing any other component. At this time, in Step S103, the solution A, the solution B, and the solution E only need to be mixed so that the blending amounts of the respective components may each be a desired value.

The number of kinds of solutions to be prepared is not limited to the example described above, and the combination thereof may be appropriately optimized in accordance with the kinds of the components to be used and the kind of the phantom to be produced.

### EXAMPLES

The present invention is described in detail below by way of Examples, but the present invention is not limited to these Examples.

### Material

Materials used in Examples and Comparative Examples are listed below. The viscosity of a material for the polysaccharide thickener (B) is a value determined by subjecting a 1 mass % aqueous solution thereof to measurement at room temperature.

### Polysaccharide (A)

A-1: agar; “Agar, powder (Kishida Chemical Co., Ltd.)”

### Polysaccharide Thickener (B)

B-1: guar gum; “CG-10 (manufactured by Ina Food Industry Co., Ltd.)”, mannose residue:galactose residue=2:1, viscosity: 213 Pa·s, number average molecular weight: about 900,000
B-2: guar gum; “CG-100 (manufactured by Ina Food Industry Co., Ltd.)”, mannose residue:galactose residue=2:1, viscosity: 1,877 Pa·s, number average molecular weight: about 1200,000
B-3: guar gum; “CG-500 (manufactured by Ina Food Industry Co., Ltd.)”, mannose residue:galactose residue=2:1, viscosity: 4,118 Pa·s, number average molecular weight: about 1500,000
B-4: Hydroxypropyl guar; “ESAFLOR 4W (manufactured by Sansho Co., Ltd.)”, mannose residue:galactose residue=2:1, viscosity: 4,734 Pa·s

### Sound Speed-Adjusting Agent

C-1: urea (manufactured by Kishida Chemical Co., Ltd.)

### Other Component

D-1 (ultrasonic scattering agent): graphite; “NICABEADS (trademark) ICB1020 (manufactured by Nippon Carbon Co., Ltd.)”
D-2 (antiseptic): methyl parahydroxybenzoate (manufactured by Kishida Chemical Co., Ltd.)
D-3 (crosslinking agent): borax (manufactured by Kishida Chemical Co., Ltd.)
D-4 (viscosity modifier): glycerin

### Evaluation Method

### Young's Modulus and Viscosity

Measurement was performed with a viscoelasticity-measuring apparatus (MCR302 manufactured by Anton Paar Japan K.K.), and the values of a storage modulus G′ and a tan δ were determined. The storage modulus G′ was measured by using a phantom having a columnar shape produced into a diameter of 30 mm and a thickness of 5 mm was used. The storage modulus G′ was measured by bringing the phantom into contact with parallel plates each having a diameter of 25 mm from above and applying a dynamic strain thereto.

The Young's modulus E of the phantom is calculated from the storage modulus G′ by using its Poisson's ratio v in accordance with the following equation.

$$E = {G}^{′} \cdot 2 ( 1 + ν )$$

The Poisson's ratio ν of the phantom can be assumed to be 0.5 because a change in volume thereof at the time of the measurement is small. In view of the foregoing, E=3·G′ was established, and hence the value of the Young's modulus E thereof was obtained by multiplying the value of the storage modulus G′ thereof by 3.

The tan δ of the phantom is the ratio of the loss modulus G″ thereof to the storage modulus G′ thereof, and is calculated from the following equation.

$$tan δ = {G}^{″} / {G}^{′}$$

The value of the tan δ has the following meaning: as the value becomes closer to 1, the phantom becomes gel having a higher viscous property, and when the value becomes more than 1, the phantom becomes sol.

### Sound Speed

A sound speed was measured by measuring a time period required for an ultrasonic wave to pass through the phantom. The phantom was fixed in water with a jig between a transducer having a measurement frequency of 3.5 MHz (V328-SU manufactured by Olympus Corporation) and a needle-type hydrophone (manufactured by Toray Engineering D Solutions Co., Ltd.). The phantom was fixed so that the angle of incidence of an ultrasonic signal became 0°.

Under a state in which test pieces each measuring 100 mm long by 100 mm wide by 5 mm or 10 mm thick were each set as described above, a 3-cycle sine wave generated by a waveform generator was oscillated from an ultrasonic oscillator, and waveform data received by an ultrasonic receiver was measured with an oscilloscope.

### Method of Producing Phantom

### Production Example

The production procedure of Example 1 is described as an example of a production procedure for a phantom. In each of the other examples and production examples, a phantom was produced while only the kinds and amounts of materials were changed in accordance with a formulation shown in Table 1 without any change of the procedure.

The solution A containing the polysaccharide (A), the solution B containing the polysaccharide thickener (B), and the solution E containing any other component were each prepared, and then the solutions were mixed to produce the phantom.

### Solution A

50 Grams (10 parts by mass) of ion-exchanged water, 1.75 g (0.35 part by mass) of agar, and 1.25 g (0.25 part by mass) of methyl parahydroxybenzoate were loaded into a closed container, and the mixture was heated with a heating oven at 90° C. for 4 hours to provide the solution A.

In Examples 6 and 7, and Comparative Example 3, 0.25 g (0.05 part by mass), 0.5 g (0.1 part by mass), and 1 g (0.2 part by mass) of borax were further loaded into solutions before their heating, respectively.

### Solution B

300 Grams (60 parts by mass) of ion-exchanged water and 15.2 g (3.04 parts by mass) of the guar gum (B-2) were loaded into a closed container, and the mixture was sufficiently stirred, followed by heating with a heating oven at 90° C. for 24 hours. Thus, the solution B was obtained.

### Solution E

In a closed container, 30 g (6 parts by mass) of urea was dissolved in 120 g (24 parts by mass) of ion-exchanged water to prepare a 20 mass % urea solution. 2.5 Grams (0.5 part by mass) of the guar gum (B-2) was added to and dissolved in the resultant urea solution. Subsequently, 20 g (4 parts by mass) of graphite was further added to the above-mentioned urea solution, and the mixture was sufficiently stirred, followed by heating with a heating oven at 90° C. for 2 hours. Thus, the solution E was obtained.

The solution A, the solution B, and the solution E were mixed at a ratio corresponding to a ratio among their respective constituent components under a state in which the solutions were heated at 90° C., and the mixture was subjected to stirring treatment for 2 minutes to provide the preparation liquid C. After that, the resultant preparation liquid C was poured into a mold, and was cooled to provide the phantom.

TABLE 1

| | | | Example | Example | Example | Example | Example | Example | Example |
|---|---|---|---|---|---|---|---|---|---|
| | | | 1 | 2 | 3 | 4 | 5 | 6 | 7 |
| Material | Water | | 94 | 94 | 94 | 94 | 94 | 94 | 94 |
| blending | Polysaccharide | A-1 | 0.35 | 0.35 | 0.5 | 0.2 | 0.01 | 0.1 | 0.1 |
| amount | (A) | | | | | | | | |
| (part(s) | Polysaccharide | B-1 | — | — | — | — | — | — | — |
| by | thickener (B) | B-2 | 3.54 | — | — | — | — | — | — |
| mass) | | B-3 | — | 3.54 | — | — | — | — | — |
| | | B-4 | — | — | 3.54 | 6.0 | 6.0 | 6.0 | 6.0 |
| | Sound speed- | C-1 | 6 | 6 | 6 | 6 | 6 | 6 | 6 |
| | adjusting agent | | | | | | | | |
| | Other | D-1 | 4 | 4 | 4 | 4 | 4 | 4 | 4 |
| | component | D-2 | 0.25 | 0.25 | 0.25 | 0.25 | 0.25 | 0.25 | 0.25 |
| | | D-3 | — | — | — | — | — | 0.05 | 0.1 |
| | | D-4 | — | — | — | — | — | — | — |

| | | | | | | | | |
|---|---|---|---|---|---|---|---|---|
| Physical | Young's modulus (kPa) | 2.8 | 3.1 | 2.6 | 5.3 | 4.4 | 5.4 | 4.8 |
| property | Viscous property: tanδ | 0.28 | 0.35 | 0.24 | 0.78 | 0.85 | 0.21 | 0.2 |
| of gel | (—) | | | | | | | |
| | Sound speed (m/s) | 1,535 | 1,536 | 1,539 | 1,532 | 1,538 | 1,542 | 1,541 |

| | | | Comparative | Comparative | Comparative |
|---|---|---|---|---|---|
| | | | Example 1 | Example 2 | Example 3 |
| Material | Water | | 94 | 94 | 94 |
| blending | Polysaccharide | A-1 | 0.35 | 0.35 | 0.1 |
| amount | (A) | | | | |
| (part(s) | Polysaccharide | B-1 | 3.54 | — | — |
| by | thickener (B) | B-2 | — | — | — |
| mass) | | B-3 | — | — | — |
| | | B-4 | — | — | 6.0 |
| | Sound speed- | C-1 | 6 | — | 6 |
| | adjusting agent | | | | |
| | Other | D-1 | 4 | 4 | 4 |
| | component | D-2 | 0.25 | 0.25 | 0.25 |
| | | D-3 | — | — | 0.2 |
| | | D-4 | — | 10 | — |

| | | | | |
|---|---|---|---|---|
| Physical | Young's modulus (kPa) | 2.4 | 1.3 | 7.2 |
| property | Viscous property: tanδ | 0.1 | 0.04 | 0.14 |
| of gel | (—) | | | |
| | Sound speed (m/s) | 1,530 | 1,530 | 1,541 |

When the polysaccharide thickener B-1 whose 1 mass % aqueous solution had a viscosity at room temperature of 213 mPa·s, that is, the guar gum CG-10 was used, tan δ≥0.2 was not satisfied, and hence a phantom satisfying the viscous property condition was not obtained. Meanwhile, when the polysaccharide thickeners B-2 to B-4 whose 1 mass % aqueous solutions each had a viscosity at room temperature of more than 300 mPa·s were each used, tan δ≥0.2 was satisfied, and hence a phantom satisfying the viscous property condition was able to be obtained. In addition, the sound speeds in the phantoms according to Examples were each a value close to a sound speed (1,535 m/s) in a living organism, and hence phantoms each having a high viscous property while suppressing a divergence from the sound speed in the living organism were able to be obtained. In addition, also when chemical crosslinking with borax was performed, tan δ≥0.2 was satisfied, and hence a phantom satisfying the viscous property condition was able to be obtained.

With regard to the results obtained in Examples 1 and 2, and Comparative Example 1, the viscosity of a 1 mass % aqueous solution of the polysaccharide thickener (B) at room temperature is plotted against an axis of abscissa, and the viscous property tan δ is plotted against an axis of ordinate to provide a scatter plot. Next, the three points in the scatter plot are approximated by a least-squares method to provide a straight line. It is understood from the straight line thus obtained that when the viscosity of the 1 mass % aqueous solution at room temperature is 300 mPa·s or more, an ultrasound phantom showing a tan δ of 0.2 or more under a gel state is obtained. The tan δ of such hydrogel is obtained by dynamic viscoelasticity measurement under room temperature at 1 Hz.

Thus, a phantom that had largely alleviated the problems of the related-art phantom was able to be obtained by using the polysaccharide (A) having a sol-gel phase transition point of 30° C. or more and 95° C. or less, and the polysaccharide thickener (B) whose 1 mass % aqueous solution had a viscosity at room temperature of 300 mPa·s or more.

According to the present invention, there can be provided the ultrasound phantom that can have a high viscous property while suppressing a divergence from a sound speed in a living organism.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2023-039061, filed Mar. 13, 2023, and Japanese Patent Applications No. 2024-027845, filed Feb. 27, 2024, which are hereby incorporated by reference herein in their entirety.

What is claimed is:

1. An ultrasound phantom comprising:
water;
a polysaccharide (A) having a sol-gel phase transition point of 30° C. or more and 95° C. or less; and
a polysaccharide thickener (B) whose 1 mass % aqueous solution has a viscosity at room temperature of 300 mPa·s or more,
wherein the ultrasound phantom is configured to show a tan δ of 0.2 or more under a gel state, which is obtained by dynamic viscoelasticity measurement at 1 Hz and room temperature.

2. The ultrasound phantom according to claim 1, wherein the polysaccharide (A) is agar.

3. The ultrasound phantom according to claim 1,
wherein the polysaccharide thickener (B) is a polysaccharide thickener having a mannose residue in a main chain thereof, and having a galactose residue in a side chain thereof, and
wherein a ratio of the number of the galactose residues present in the side chain to the number of the mannose residues present in the main chain is 0.2 or more and 0.4 or less.

4. The ultrasound phantom according to claim 1, wherein the polysaccharide thickener (B) has a molecular weight of 90,000 or more.

5. The ultrasound phantom according to claim 1, wherein the polysaccharide thickener (B) contains a polysaccharide thickener selected from a guar gum, a locust bean gum, and a tamarind seed gum.

6. The ultrasound phantom according to claim 1, further comprising a sound speed-adjusting agent.

7. The ultrasound phantom according to claim 1, further comprising an ultrasonic scattering agent.

8. The ultrasound phantom according to claim 1, further comprising an antiseptic.

9. A method of producing an ultrasound phantom, the method comprising:
preparing a solution A containing a polysaccharide (A) having a sol-gel phase transition point of 30° C. or more and 95° C. or less;
preparing a solution B containing a polysaccharide thickener (B) whose 1 mass % aqueous solution has a viscosity at room temperature of 300 mPa·s or more;
preparing a preparation liquid C by mixing the solution A and the solution B; and
preparing a hydrogel D by causing the preparation liquid C to gelate,
wherein the hydrogel D in a gel state shows a tan δ of 0.2 or more, which is obtained by performing dynamic viscoelasticity measurement at 1 Hz and room temperature.

10. The method of producing an ultrasound phantom according to claim 9, wherein the preparing the hydrogel D includes cooling the preparation liquid C.
